(19) United States
(12) Patent Application Publication
(10) Pub. No.: US 2025/0019281 A1
(43) Pub. Date: Jan. 16, 2025

(54) MICROBIAL COMPOSITE REAGENT AND PREPARATION METHOD THEREOF, AND METHOD FOR REMOVING PERFLUOROOCTANOIC ACID

(71) Applicant: TSINGHUA UNIVERSITY, BEIJING (CN)

(72) Inventors: Deyi HOU, BEIJING (CN); Kaikai ZHANG, BEIJING (CN); Jiayu DENG, BEIJING (CN); Ying HU, BEIJING (CN)

(73) Assignee: TSINGHUA UNIVERSITY, BEIJING (CN)

(21) Appl. No.: 18/896,948

(22) Filed: Sep. 26, 2024

Related U.S. Application Data
(63) Continuation-in-part of application No. PCT/CN2024/072900, filed on Jan. 18, 2024.

(30) Foreign Application Priority Data
Apr. 24, 2023 (CN) 202310449642.9

(51) Int. Cl. C02F 3/34; C02F 101/36; C12N 1/38; C12R 1/145

(52) U.S. Cl. CPC C02F 3/34; C12N 1/38; C02F 2101/36; C12R 2001/145

(57) ABSTRACT

A microbial composite reagent, a preparation method thereof, and a method for removing perfluorooctanoic acid are disclosed. The preparation method of the microbial composite reagent includes steps of: preparing a glucose-based carbonaceous material by pyrolyzing glucose at 400° C. to 1000° C. for at least 0.5 hours under a protective atmosphere; mixing perfluorooctanoic acid-contaminated soil with a basal medium, molasses, a vitamin B, and water, and subjecting the mixture to microbial acclimation under an anaerobic condition to obtain an acclimated microorganism solution; subjecting the glucose-based carbonaceous material to microbial loading by anaerobically mixing the glucose-based carbonaceous material with the acclimated microorganism solution, thereby obtaining the microbial composite reagent.

### CROSS-REFERENCE TO RELATED APPLICATIONS

This application is a continuation in part of international patent application No. PCT/CN2024/072900, filed on Jan. 18, 2024, which itself claims priority of China Patent Application No. 202310449642.9, filed on Apr. 24, 2023, entitled “MICROBIAL COMPOSITE REAGENT AND PREPARATION METHOD THEREOF, AND METHOD FOR REMOVING PERFLUOROOCTANOIC ACID”. The contents of the above identified applications are hereby incorporated herein in their entireties by reference.

### TECHNICAL FIELD

The present application relates to the field of organic pollutant treatment, and in particular to a microbial composite reagent, a preparation method thereof, and a method for removing perfluorooctanoic acid.

### BACKGROUND

Since the 1940s and 1950s, perfluoroalkyl and polyfluoroalkyl substances (PFASs) have been widely used in people's work and life, as well as in various industrial and consumer products. As an important fluorinated surfactant, perfluorooctanoic acid (PFOA) is one of the most commonly used PFASs today, with widespread applications in both industrial and household products. However, the extensive use of PFOA has led to environmental contamination. PFOA can be detected across diverse environmental media, including oceans, rivers, lakes, groundwater, and soil, and also can be found in both wild animals and humans. With a long half-life of up to 3.8 years in the human body, PFOA tends to accumulate in the liver and blood, posing health risks such as reproductive toxicity, an increased risk of cancer, and adverse effects on reproduction and genetics. These risks directly threaten human survival, reproduction, and sustainable development. Therefore, there is an urgent need to eliminate PFASs, including PFOA, from environmental media.

PFOA has excellent thermal and chemical stability due to the highly polarized C-F bonds in the molecular structure thereof with bond energy up to 485 KJ/mol. Currently, the primary methods for treating PFOA-contaminated soil involve excavation and off-site disposal. For PFOA-contaminated groundwater, extraction methods are mainly employed. Additionally, adsorbents based on activated carbon materials and ion exchange resins can be used to treat PFOA-contaminated soil and groundwater; however, PFOA adsorbed onto these materials may still be released back into the environment under changing conditions, leading to secondary pollution. Chemical oxidation techniques based on Fenton reactions, persulfates, aeration, and potassium permanganate have been proven ineffective against PFOA and similar compounds. Modified reduction technologies based on nano zero-valent iron (nZVI) can reduce PFOA by generating reactive hydrogen species on the catalyst surface, but nZVI has a limited application range and is less effective against PFOA and similar compounds under near-neutral conditions. Chemical techniques based on UV irradiation and electrochemistry involve stringent operational conditions and high costs, making them unsuitable for the remediation of PFOA in soil and groundwater. Microbial bioremediation technologies, on the other hand, offer advantages such as low cost and simplicity of operation.

### SUMMARY

In view of the above, there is a need to provide a microbial composite reagent, a preparation method thereof, and a method for removing perfluorooctanoic acid.

A method for preparing a microbial composite reagent includes the steps of:
preparing a glucose-based carbonaceous material by pyrolyzing glucose at a temperature in a range from 400° C. to 1000° C. for at least 0.5 hours under a protective atmosphere;
mixing perfluorooctanoic acid-contaminated soil with a basal medium, molasses, a vitamin B, and water, and subjecting the mixture to microbial acclimation under an anaerobic condition to obtain an acclimated microorganism solution; and
subjecting the glucose-based carbonaceous material to microbial loading by anaerobically mixing the glucose-based carbonaceous material with the acclimated microorganism solution, thereby obtaining the microbial composite reagent.

In some embodiments, in the step of preparing the glucose-based carbonaceous material, the pyrolysis lasts for 2 hours to 3 hours.

In some embodiments, a concentration of perfluorooctanoic acid in the perfluorooctanoic acid-contaminated soil is greater than 0 and less than or equal to 0.5 mg/L.

In some embodiments, corresponding to every 10 parts by mass of the perfluorooctanoic acid-contaminated soil, an amount of the basal medium is in a range from 9 parts by mass to 18 parts by mass, an amount of molasses is in a range from 0.5 parts by mass to 5 parts by mass, an amount of the vitamin B is in a range from 0.1 parts by mass to 1 part by mass, and an amount of water is in a range from 500 parts by mass to 1000 parts by mass.

In some embodiments, the vitamin B includes vitamin B12.

In some embodiments, the basal medium includes a nutrient broth.

In some embodiments, the microbial acclimation lasts for 5 days to 10 days, and a temperature of the microbial acclimation is in a range from 20° C. to 35° C.

In some embodiments, a concentration of the glucose-based carbonaceous material in the microbial composite reagent is in a range from 0.1 g/L to 1 g/L.

In some embodiments, the microbial loading lasts for 3 days to 7 days.

A microbial composite reagent includes a glucose-based carbonaceous material and an acclimated microorganism solution. The microorganism solution includes perfluorooctanoic acid-contaminated soil, a basal medium, molasses, a vitamin B, and water.

A specific surface area of the glucose-based carbonaceous material is in a range from 10.817 m²/g to 105.719 m²/g, an intensity ratio of the D peak to the G peak in a Raman spectrum of the glucose-based carbonaceous material is in a range from 0.63 to 1.20.

In some embodiments, a concentration of the glucose-based carbonaceous material in the microbial composite reagent is in a range from 0.1 g/L to 1 g/L.

In some embodiments, a concentration of perfluorooctanoic acid in the perfluorooctanoic acid-contaminated soil is greater than 0 and less than or equal to 0.5 mg/L.

In some embodiments, corresponding to every 10 parts by mass of the perfluorooctanoic acid-contaminated soil, an amount of the basal medium is in a range from 9 parts by mass to 18 parts by mass, an amount of molasses is in a range from 0.5 parts by mass to 5 parts by mass, an amount of the vitamin B is in a range from 0.1 parts by mass to 1 part by mass, and an amount of water is in a range from 500 parts by mass to 1000 parts by mass.

In some embodiments, the vitamin B includes vitamin B12.

In some embodiments, the basal medium includes a nutrient broth.

In some embodiments, the microbial composite reagent is prepared by the above-described method for preparing the microbial composite reagent.

A method for removing perfluorooctanoic acid includes a step of treating a pollution source with a microbial composite reagent to remove perfluorooctanoic acid from the pollution source. The microbial composite reagent is prepared by the above-described method for preparing the microbial composite reagent, or is the above-described microbial composite reagent.

In some embodiments, an initial concentration of perfluorooctanoic acid in the pollution source is in a range from 0.1 mg/L to 5 mg/L.

In some embodiments, the pollution source includes contaminated soil and/or contaminated groundwater; a volume ratio of the microbial composite reagent to the pollution source is in a range from 1:10 to 1:20.

In some embodiments, a time period of treating the pollution source with the microbial composite reagent is greater than 20 days.

### BRIEF DESCRIPTION OF THE DRAWINGS

To describe the technical solutions in the embodiments of the present application or in the conventional art more clearly, the following briefly introduces the accompanying drawings required for describing the embodiments or the conventional art. Apparently, the following described drawings only illustrate embodiments of the present application. For a person of ordinary skill in the art, other related drawings can also be obtained according to the following drawings without creative efforts.

FIG. 1 shows a flow chart of a method for preparing a microbial composite reagent according to an embodiment.

FIGS. 2A and 2B show XRD and Raman spectra of glucose-based carbonaceous materials prepared in Examples 1 to 3.

FIG. 3 shows comparison of PFOA removal rates after treating PFOA-contaminated soil and groundwater for 40 days with: microorganisms (M) alone in Comparative Example 1, a glucose-based carbonaceous material (BC) alone in Comparative Example 2, and microbial composite reagents (M+BC) in Examples 1 to 3.

FIG. 4 shows concentration change curves of PFOA in reaction systems with different treatment time periods and glucose-based carbonaceous materials obtained at different pyrolysis temperatures, under actions of microorganisms alone in Comparative Example 1 or microbial composite reagents in Examples 1 to 3.

FIG. 5 shows concentration change curves of fluoride ions in reaction systems with different treatment times and glucose-based carbonaceous materials obtained at different pyrolysis temperatures, under actions of microorganisms alone (M) in Comparative Example 1 or microbial composite reagents in Examples 1 to 3.

FIG. 6 shows comparison of PFOA removal effects of microbial composite reagents obtained in Example 1, Example 4, and Example 5, step (3).

FIG. 7 shows comparison of PFOA removal effects of microbial composite reagents obtained in steps (3) of Example 2, Example 6, and Example 7.

FIG. 8 shows comparison of PFOA removal effects of microbial composite reagents obtained in steps (3) of Example 3, Example 8, and Example 9.

FIG. 9 shows comparison of PFOA removal effects of microorganisms alone in Comparative Example 1 and a blank control without microorganisms.

FIG. 10 shows comparison of PFOA removal effects of microbial composite reagents prepared in steps (3) of Comparative Example 3 and Comparative Example 4.

FIG. 11 shows comparison of PFOA removal effects of microbial composite reagents prepared in steps (3) of Comparative Example 5 and Comparative Example 6.

FIGS. 12A to 12C show a microbial community structure and relative abundances in systems under actions of microorganisms alone (M) in Comparative Example 1 and microbial composite reagents in Example 1, Example 2, and Example 3.

### DETAILED DESCRIPTION

In order to facilitate the understanding of the present application, the present application will now be comprehensively described with reference to specific implementation modes. Embodiments of the present application are provided in the specific implementation modes. However, the present application can be implemented in many different forms and therefore is not limited to the embodiments described herein. On the contrary, the purpose of providing these embodiments is to provide a more thorough and comprehensive understanding of the disclosed content of the present application.

Unless otherwise defined, all technical and scientific terms used herein have the same meanings as commonly understood by a person skilled in the art to which the present application pertain. Additionally, the terms used in the description of the present application are merely for describing the specific embodiments, and are not intended to limit the present application.

Unless otherwise stated or there is a contradiction, the terms or phrases used in the present application have the following meanings.

In the present application, “first” and “second” are only for descriptive purposes and should not be understood as indicating or implying relative importance, nor as implying the quantity of the indicated technical features. Thus, the feature modified with “first” or “second” may explicitly or implicitly include at least one such feature.

In the present application, “at least one” refers to any one, two, or more items in a list.

In the present application, when a concentration in percentage is mentioned, unless otherwise specifically stated, it always refers to the final concentration, referring to the proportion of the added component in a system after adding that component.

In the present application, the terms such as “preferably” and “more preferably” refer to certain beneficial effects that may be provided in certain embodiments of the present application in certain circumstances. However, other embodiments may be also preferable in the same or other circumstances. Furthermore, the expression of one or more preferred embodiments does not imply that other embodiments are not applicable, nor is it intended to exclude other embodiments from the scope of the present application.

In the present application, when a numerical range is disclosed, the range is considered to be continuous and includes the minimum value and the maximum value of the range, as well as each value between the minimum value and the maximum value. Further, when the range is for integers, it includes each integer between the minimum value and maximum value of the range. In addition, when multiple ranges are provided to describe a characteristic or feature, those ranges can be combined. In other words, unless otherwise stated, all ranges disclosed in the present application should be understood to include any and all sub-ranges included therein.

In the present application, an open-ended description for the technical features includes not only a close-ended technical solution consisting of the recited technical features, but also an open-ended technical solution including the recited technical features.

In the embodiments of the present application, the terms “including”, “having” and any variations thereof are intended to cover non-exclusive inclusion. For example, a process, a method, a system, a product, or a device including a series of steps or units is not limited to the listed steps or units, but optionally also includes steps or units that are not listed, or optionally includes other steps or components that are inherent to the process, the method, the product, or the device.

In the present application, the term “embodiment” means that specific features, structures, or characteristics described with reference to the embodiment can be included in at least one embodiment of the present application. This phrase appeared at various places in the specification does not necessarily refer to the same embodiment, nor are they mutually exclusive independent or alternative embodiments. It can be understood by those skilled in the art, both explicitly and implicitly, that the embodiments described in the present application may be combined with other embodiments.

In the related art, microbial anaerobic fermentation treatment technique has been used to promote the biodegradation of perfluorinated compounds in sludge or sediment to achieve degradation of the perfluorinated compounds during microbial anaerobic fermentation. Specifically, the sludge or sediment with its pH adjusted to alkaline is added with a surfactant, and subjected to anaerobic fermentation to degrade the perfluorinated compounds. The surfactant is added at a mass ratio of (0.05-0.50):1 relative to the dry weight of the sludge/sediment sludge, which can effectively destroy the extracellular polymeric substances in the sludge or sediment and allow a large amount of perfluorinated compound pollutants to be desorbed into the liquid solution. The degradation rate of the perfluorinated compounds is over 20%, with a maximum of 56.3%. However, in this method, the addition of the surfactant increases the cost, and may cause secondary pollution. Moreover, the biodegradation of the perfluorinated compounds in the aqueous phase is not addressed, and the degradation rate of the perfluorinated compounds is relatively low.

In the related art, electron acceptors can also be used to promote the degradation of perfluorinated compounds. One or more electron acceptors selected from manganese ions, nitrate ions, sulfate ions, iron ions, and methane can be added into the perfluorinated compound contaminated sediment to promote the biodegradation and adsorption of the perfluorinated compounds in the natural sediment. However, this method mainly targets the perfluorinated compounds in the sediment and has relatively low degradation efficiency. Moreover, the proportion of the added electron acceptors can be as high as 5%, which may cause serious secondary pollution problems.

In the related art, the anaerobic feammox under anaerobic conditions can also be used to achieve the degradation of perfluorinated compounds, which mainly involves adding iron ions and dissimilatory iron-reducing bacteria and introducing electron donors into the contaminated sediment to achieve the degradation of the perfluorinated compounds. However, the added amount of the electron donors is relatively high, ranging from 5% to 10%, resulting in a relatively high COD content in the sludge. Moreover, although the anaerobic feammox process can degrade the perfluorinated compounds within 20 days, its actual effectiveness is relatively poor, not suitable for the remediation and treatment of contaminated soil and groundwater.

In the related art, the sludge-based biochar can also be used as an adsorbent to remove PFOS and PFOA from water through adsorption. The sludge-based biochar is prepared by a method involving drying, activation, high temperature carbonization treatment, acid leaching, washing, and oven-drying. However, the preparation process of the sludge-based biochar is complex, and the sludge-based biochar only targets low concentrations of PFOA (ranging from 0.01 mg/mL to 1.1 mg/L) in water with a maximum removal rate of 71%, and cannot be used to remove PFOA from soil or for higher concentrations of PFOA from water bodies.

Due to the issues, such as the low degradation efficiency and the risk of secondary pollution, associated with the microbe-based bioremediation techniques in the related art, there is a promising application potential for developing an enhanced bioremediation technology for remediating PFOA-contaminated soil and groundwater. In view of this, embodiments of the present application provide a method for preparing a microbial composite reagent based on a glucose-based carbonaceous material in combination with microorganisms.

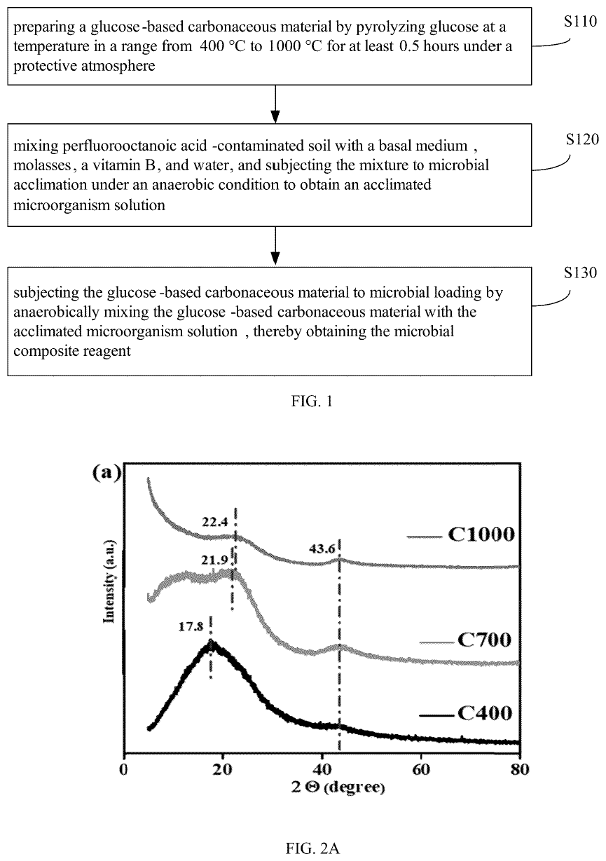

Referring to FIG. 1, a method for preparing a microbial composite reagent includes following steps S110 to S130.

Step S110, a glucose-based carbonaceous material is prepared by pyrolyzing glucose at a temperature in a range from 400° C. to 1000° C. for at least 0.5 hours under a protective atmosphere.

In some embodiments, the protective atmosphere can be, but is not limited to, nitrogen gas or other commonly used protective gases, such as argon gas. It should be understood that being under a protective atmosphere can be directly in an atmosphere full of the protective gas, or in a system to which the protective gas is continuously introduced.

In some embodiments, a flow rate of the protective gas is in a range from 0.5 mL/min to 2 mL/min. The flow rate of the protective gas can be, for example, but is not limited to, 0.5 mL/min, 0.6 mL/min, 0.8 mL/min, 1 mL/min, 1.2 mL/min, 1.4 mL/min, 1.5 mL/min, 1.6 mL/min, 1.8 mL/min, 2 mL/min, or in any range defined between any two of these values.

In some embodiments, the temperature of pyrolysis can be, but is not limited to, 400° C., 450° C., 500° C., 550° C., 600° C., 650° C., 700° C., 750° C., 800° C., 850° C., 900° C., 950° C., 1000° C., or in any range defined between any two of these values. Optionally, the temperature of pyrolysis is 400° C. to 700° C., 500° C. to 900° C., 600° C. to 1000° C., or 700° C. to 1000° C. It has been proven by experiments that if the temperature of pyrolysis is too low, the achieved glucose-based carbonaceous material has relatively poor electrical conductivity and relatively weak electron transport capability, leading to reduced effectiveness in promoting the micro-biological degradation of PFOA. If the temperature of pyrolysis is too high, the achieved glucose-based carbonaceous material has a relatively small specific surface area, resulting in limited microbial loading capability and limited effects on improving the degradation efficiency of PFOA.

In some embodiments, the time period of pyrolysis can be, but is not limited to, 0.5 h, 1 h, 1.5 h, 2 h, 2.5 h, 3 h, 3.5 h, 4 h, 4.5 h, 5 h, or in any range defined between any two of these values. Further, the time period of pyrolysis is ranged from 2 h to 3 h.

In some embodiments, the heating rate is in a range from 5° C./min to 15° C./min. For example, the heating rate can be, but is not limited to, 5° C./min, 6° C./min, 7° C./min, 8° C./min, 9° C./min, 10° C./min, 11° C./min, 12° C./min, 13° C./min, 14° C./min, 15° C./min, or in any range defined between any two of these values.

In some embodiments, step S110 includes: under a protective gas with a flow rate of 0.5 mL/min to 2 mL/min, heating the glucose to a temperature of 400° C. to 1000° C. at a heating rate of 5° C./min to 15° C./min and keeping the glucose at this temperature for 2 h to 3 h to prepare the glucose-based carbonaceous material.

In this embodiment, through the pyrolysis of glucose at the high temperature, a glucose-based carbonaceous material with good electrical conductivity, good electron transport capability, and good microbial loading capability can be obtained. The preparation method of the glucose-based carbonaceous material is simple, and through the synergy of the glucose-based carbonaceous material and microorganisms, the degradation of PFOA in soil and groundwater is realized with a high degradation rate. In addition, glucose is a monosaccharide and can also provide nutrients that can be directly used for the growth of microorganisms.

Step S120, perfluorooctanoic acid-contaminated soil is mixed with a basal medium, a molasses, a vitamin B, and water, and the mixture is subjected to microbial acclimation under an anaerobic condition to obtain an acclimated microorganism solution. The perfluorooctanoic acid-contaminated soil includes fermentative electroactive microorganisms belonged to Clostridia (class), specifically includes microorganisms belonged to Dehalobacter (genus) and/or microorganisms belonged to Desulfovibrio (genus), and can further optionally includes microorganisms belonged to Sedimentibacter (genus), Clostridium_sensu_stricto_8 (genus), Azobacter (genus), Fonticella (genus), or any combinations thereof. Among them, Dehalobacter and Desulfovibrio can transfer electrons to PFOA under the action of the glucose-based carbonaceous material, thereby promoting the reductive dehalogenation of PFOA. Additionally, Sedimentibacter, Clostridium_sensu_stricto_8, Azobacter, Fonticella are able to provide electrons for the metabolism of Dehalobacter and Desulfovibrio, thus promoting the growth of these dehalogenating bacteria. It has been found through experiments that as long as the soil contains these microorganisms, it meet the basic requirements for the microorganisms in the soil used in the microbial composite agent described in the present application. The anaerobic environment and perfluorooctanoic acid in perfluorooctanoic acid-contaminated soil are particularly conducive to the growth and reproduction of these microorganisms. However, the above described microorganisms indeed widely exist in natural soils and waters, and thus the source of soil is not limited. In the examples and comparative examples described below, the soils were collected from the land of woods and waters at Tsinghua University.

In some embodiments, a concentration of perfluorooctanoic acid in the perfluorooctanoic acid-contaminated soil is greater than 0 and less than or equal to 0.5 mg/L, for example can be in a range from 0.001 mg/L to 0.5 mg/L, further can be in a range from 0.05 mg/L to 0.5 mg/L. For example, the concentration of perfluorooctanoic acid can be, but is not limited to, 0.001 mg/L. 0.005 mg/L. 0.01 mg/L. 0.03 mg/L. 0.05 mg/L, 0.07 mg/L, 0.1 mg/L, 0.13 mg/L, 0.17 mg/L, 0.2 mg/L, 0.23 mg/L, 0.27 mg/L, 0.3 mg/L, 0.33 mg/L, 0.37 mg/L, 0.4 mg/L, 0.43 mg/L, 0.47 mg/L, 0.5 mg/L, or in any range defined between any two of these values.

In some embodiments, the vitamin B includes, but is not limited to, vitamin B12.

In some embodiments, water used in step S120 can be, but is not limited to, deionized water.

In some embodiments, corresponding to every 10 parts by mass of the perfluorooctanoic acid-contaminated soil, an amount of the basal medium is in a range from 9 parts by mass to 18 parts by mass, an amount of molasses is in a range from 0.5 parts by mass to 5 parts by mass, an amount of the vitamin B is in a range from 0.1 parts by mass to 1 part by mass, and an amount of water is in a range from 500 parts by mass to 1000 parts by mass.

The basal medium is used to provide essential nutrients for microorganisms. Optionally, the basal medium includes a nutrient broth. In a specific example, the basal medium can be, but is not limited to, nutrient broth 022010 or other commonly used media in the art. Molasses serves as a nutrient that can be utilized by microorganisms and can enhance hydrogen production, thereby promoting reductive defluorination of PFOA through hydrogen substitution reactions. The vitamin B can promote the growth of dehalogenating microorganisms, thereby promoting the reductive dehalogenation of PFOA.

Optionally, corresponding to every 10 parts by mass of the perfluorooctanoic acid-contaminated soil, the amount by mass of the basal medium can be, but is not limited to, 9 parts, 10 parts, 11 parts, 12 parts, 13 parts, 14 parts, 15 parts, 16 parts, 17 parts, 18 parts, or in any range defined between any two of these values; the amount by mass of molasses can be, but is not limited to, 0.5 parts, 1 part, 1.5 parts, 2 parts, 2.5 parts, 3 parts, 3.5 parts, 4 parts, 4.5 parts, or in any range defined between any two of these values; the amount by mass of the vitamin B can be, but is not limited to, 0.1 parts, 0.2 parts, 0.3 parts, 0.4 parts, 0.5 parts, 0.6 parts, 0.7 parts, 0.8 parts, 0.9 parts, 1 part, or in any range defined between any two of these values; the amount by mass of water can be, but is not limited to, 500 parts, 550 parts, 600 parts, 650 parts, 700 parts, 750 parts, 800 parts, 850 parts, 900 parts, 950 parts, 1000 parts, or in any range defined between any two of these values.

In some embodiments, a time period of the microbial acclimation is in a range from 5 days to 10 days. For example, the time period of the microbial acclimation can be, but is not limited to, 5 days, 6 days, 7 days, 8 days, 9 days, 10 days, or in any range defined between any two of these values.

In some embodiments, a temperature of the microbial acclimation is in a range from 20° C. to 35° C. For example, the temperature of the microbial acclimation can be, but is not limited to, 20° C., 22° C., 25° C., 28° C., 30° C., 32° C., 35° C., or in any range defined between any two of these values.

In some embodiments, step S120 includes: adding 9 parts by mass to 18 parts by mass of the nutrient broth, 0.5 parts by mass to 5 parts by mass of molasses, 0.1 parts by mass to 1 part by mass of the vitamin B12, and 500 parts by mass to 1000 parts by mass of water into 10 parts by mass of the PFOA-contaminated soil to obtain a mixture, and subjecting the mixture to microbial acclimation under an anaerobic condition at 20° C. to 35° C. for 5 days to 10 days, wherein the concentration of PFOA in the PFOA-contaminated soil is greater than 0 and less than or equal to 0.5 mg/L.

Some researchers have conducted experiments to remove PFOA by screening PFOA-degrading strains; however, the results have been proven to be unstable with low degradation rates in practical applications, and the influence of indigenous microorganisms has not been considered. For example, in related art, one type of PFOA-degrading strain, YAB-3, is used to degrade a PFOA-containing material by the following method: firstly, a single colony of YAB-3 is inoculated into a LB medium for 12 to 18 hours, then the medium is formed into a bacterial suspension with a certain OD600, and finally the PFOA-containing material is treated with the bacterial suspension. However, in this method, the removal efficiency of PFOA by this strain is not taken into account, and the degradation process may be influenced by various indigenous microorganisms. In related art, microorganisms containing gene of another type of perfluorinated compound-degrading bacteria is used to degrade the perfluorinated compound, and the degradation efficiency can reach 75%. However, this method fails to detail the application of this bacterial gene in contaminated sites and does not consider the influence of indigenous microorganisms.

In contrast, in the present embodiment, the indigenous microorganisms in the contaminated soil are acclimated in the basal medium containing molasses and the vitamin B, followed by mixing with the glucose-based carbonaceous material to degrade PFOA, which can achieve relatively high and stable degradation efficiency.

Step S130: the glucose-based carbonaceous material is mixed with the acclimated microorganism solution, to load microorganisms under an anaerobic condition, thereby obtaining the microbial composite reagent.

In some embodiments, a concentration of the glucose-based carbonaceous material in the microbial composite reagent is in a range from 0.1 g/L to 1 g/L. For example, the concentration of the glucose-based carbonaceous material can be, but is not limited to, 0.1 g/L, 0.2 g/L, 0.3 g/L, 0.4 g/L, 0.5 g/L, 0.6 g/L, 0.7 g/L, 0.8 g/L, 0.9 g/L, 1 g/L, or in any range defined between any two of these values. Further, the concentration of the glucose-based carbonaceous material in the microbial composite reagent is in a range from 0.1 g/L to 0.5 g/L.

In some embodiments, a time period of the microbial loading is in a range from 3 days to 7 days. For example, the time period of the microbial loading can be, but is not limited to, 3 days, 3.5 days, 4 days, 4.5 days, 5 days, 5.5 days, 6 days, 6.5 days, 7 days, or in any range defined between any two of these values.

In some embodiments, step S130 includes: adding the glucose-based carbonaceous material into the acclimated microorganism solution until the glucose-based carbonaceous material is at a concentration of 0.1 g/L to 1 g/L, and subjecting the glucose-based carbonaceous material to microbial loading under the anaerobic condition for 3 days to 7 days to obtain the microbial composite reagent.

The method for preparing the microbial composite reagent provided by the embodiments of the present application has at least the following advantages.

In the above method for preparing the microbial composite reagent, glucose is pyrolyzed at a high temperature, the perfluorooctanoic acid-contaminated soil is subjected to microbial acclimation under the condition with the basal medium, molasses and the vitamin B for a certain period of time, and then the glucose-based carbonaceous material is mixed with the acclimated microorganism solution for microbial loading, thereby obtaining the microbial composite reagent. In the above method, the glucose-based carbonaceous material obtained by high-temperature pyrolysis has good electrical conductivity, good electron transport capability, and good microbial loading capability. In the process of microbial acclimation, molasses serves as a nutrient that can be utilized by the microorganisms and can enhance hydrogen production of the microorganisms, thereby promoting the reductive defluorination of PFOA through hydrogen substitution reactions. The vitamin B can promote the growth of dehalogenating microorganisms, thereby promoting the reductive dehalogenation of PFOA. By combining the glucose-based carbonaceous material with the microorganisms to prepare the composite reagent, the activity of the microorganisms and the electron conduction between the microorganisms and PFOA are improved. In the process of removing perfluorooctanoic acid from the soil and groundwater, PFOA in water can be adsorbed and removed, while PFOA in the soil can be enriched onto the glucose-based carbonaceous material, and finally PFOA can be biodegraded by the microorganisms loaded on the glucose-based carbonaceous material, thereby achieving the bioremediation of PFOA-contaminated soil and groundwater. This method reduces the secondary pollution, is friendly to the environment, and achieves a biodegradation rate of over 90%.

The above preparation method is simple, cost effective, environmentally friendly, and easy to popularize and apply.

The present application provides an embodiment of a microbial composite reagent including a glucose-based carbonaceous material and an acclimated microorganism solution. The microorganism solution includes perfluorooctanoic acid-contaminated soil, a basal medium, molasses, a vitamin B, and water.

A specific surface area of the glucose-based carbonaceous material is in a range from 10.817 m²/g to 105.719 m²/g. An intensity ratio of the D peak around 1350 cm⁻¹ of Raman shift to the G peak around 1580 cm⁻¹ of Raman shift in a Raman spectrum of the glucose-based carbonaceous material is in a range from 0.63 to 1.20. Due to including the perfluorooctanoic acid-contaminated soil, the microbial composite reagent includes fermentative electroactive microorganisms belonged to Clostridia (class), specifically includes microorganisms belonged to Dehalobacter (genus) and/or microorganisms belonged to Desulfovibrio (genus), and can further optionally includes microorganisms belonged to Clostridia (class), Sedimentibacter (genus), Clostridium_sensu_stricto_8 (genus), Azobacter (genus), Fonticella (genus), or any combinations thereof.

In some embodiments, a concentration of the glucose-based carbonaceous material in the microbial composite reagent is in a range from 0.1 g/L to 1 g/L. Further, the concentration of the glucose-based carbonaceous material in the microbial composite reagent is in a range from 0.1 g/L to 0.5 g/L

In some embodiments, a concentration of perfluorooctanoic acid in the perfluorooctanoic acid-contaminated soil is greater than 0 and less than or equal to 0.5 mg/L.

In some embodiments, corresponding to every 10 parts by mass of the perfluorooctanoic acid-contaminated soil, an amount of the basal medium is in a range from 9 parts by mass to 18 parts by mass, an amount of molasses is in a range from 0.5 parts by mass to 5 parts by mass, an amount of the vitamin B is in a range from 0.1 parts by mass to 1 part by mass, and an amount of water is in a range from 500 parts by mass to 1000 parts by mass.

Optionally, the vitamin B includes vitamin B12.

Optionally, the basal medium includes a nutrient broth.

In some embodiments, the microbial composite reagent is prepared by the above-described method for preparing the microbial composite reagent.

In the above microbial composite reagent, the glucose-based carbonaceous material has good electrical conductivity, good electron transport capability, and good microbial loading capability. In the process of microbial acclimation, molasses serves as a nutrient that can be utilized by the microorganisms and can enhance hydrogen production of the microorganisms, thereby promoting the reductive defluorination of PFOA through hydrogen substitution reactions. The vitamin B can promote the growth of dehalogenating microorganisms, thereby promoting the reductive dehalogenation of PFOA. By combining the glucose-based carbonaceous material and the microorganisms, the activity of the microorganisms and the electron conduction between the microorganisms and PFOA are improved. In the process of removing perfluorooctanoic acid from the soil and groundwater, PFOA in water can be adsorbed and removed, while PFOA in the soil can be enriched onto the glucose-based carbonaceous material, and finally can be biodegraded by the microorganisms loaded on the glucose-based carbonaceous material, thereby achieving the bioremediation of PFOA-contaminated soil and groundwater. This method reduces the secondary pollution, is friendly to the environment, and achieves a biodegradation rate of over 90%.

The present application provides an embodiment of a method for removing perfluorooctanoic acid, including a step of treating a pollution source with a microbial composite reagent to remove perfluorooctanoic acid from the pollution source.

Specifically, the microbial composite reagent is the microbial composite reagent in the above-described embodiments or the microbial composite reagent prepared by the method for preparing the microbial composite reagent in the above-described embodiments.

In some embodiments, the pollution source includes contaminated soil and/or contaminated groundwater. A volume ratio of the microbial composite reagent to the pollution source is in a range from 1:10 to 1:20. For example, the volume ratio of the microbial composite reagent to the pollution source can be, but is not limited to, 1:10, 1:12, 1:14, 1:16, 1:18, 1:20, or in any range defined between any two of these values. It should be understood that the pollution source is not limited to the contaminated soil and the contaminated groundwater.

In some embodiments, an initial concentration of PFOA in the pollution source is in a range from 0.1 mg/L to 5 mg/L. For example, the initial concentration of PFOA can be, but is not limited to, 0.1 mg/L, 0.3 mg/L, 0.7 mg/L, 1 mg/L, 1.3 mg/L, 1.7 mg/L, 2 mg/L, 2.3 mg/L, 2.7 mg/L, 3 mg/L, 3.3 mg/L, 3.7 mg/L, 4 mg/L, 4.3 mg/L, 4.7 mg/L, 5 mg/L, or in any range defined between any two of these values. Further, the initial concentration of PFOA in the pollution source is in a range from 1 mg/L to 5 mg/L.

In some embodiments, a time period of treating the pollution source with the microbial composite reagent is greater than 20 days. For example, the time period of treating the pollution source with the microbial composite reagent can be, but is not limited to, 25 days, 30 days, 35 days, 40 days, 45 days, 50 days, 55 days, 60 days, 65 days, 70 days, 75 days, 80 days, or in any range defined between any two of these values. Further, the time period of treating the pollution source with the microbial composite reagent is less than 90 days.

To address the issues in remediation of PFOA-contaminated soil and groundwater, such as limited available technologies, high costs, and low bioremediation efficiency, in the present embodiment, the biodegradation efficiency of PFOA is enhanced by preparing a high-performance glucose-based carbonaceous material, thereby achieving the biodegradation of PFOA in contaminated soil and groundwater. By employing the treatment method combining the glucose-based carbonaceous material with the microorganisms, PFOA in water can be adsorbed and removed, while PFOA in the soil can be enriched onto the glucose-based carbonaceous material, and finally PFOA can be biodegraded by the microorganisms loaded on the glucose-based carbonaceous material.

The microbial composite reagent, the method for removing perfluorooctanoic acid, and the effects thereof in the present application will be further described in detail below in conjunction with specific examples. It should be understood that the specific examples described herein are only used to illustrate the present application and are not intended to limit the present application. Unless otherwise specifically stated, the following examples do not include other components except for unavoidable impurities. Unless otherwise specifically stated, the substances and devices used in the examples are common selections in the art. Experimental methods for which specific conditions are not indicated in the examples shall be implemented in accordance with conventional conditions, such as those described in the literature, in books, or as recommended by the manufacturers.

### Example 1

This example provides a method for removing perfluorooctanoic acid from soil and groundwater, including the following steps:

(1) Under a nitrogen flow rate of 0.5 mL/min, glucose powder is heated to 400° C. at 5° C./min for pyrolysis, and kept at 400° C. for 2 hours to obtain a glucose-based carbonaceous material.

(2) PFOA is added to soil collected from the land of woods and waters at Tsinghua University until the PFOA concentration in the soil reaches 0.01 mg/L, thereby obtaining a PFOA-contaminated soil. 10 g of the PFOA-contaminated soil with the PFOA concentration of 0.01 mg/L is added with 9 g of nutrient broth 022010, 1 g of molasses, 0.3 g of vitamin B12, and 500 mL of deionized water, and subjected to microbial acclimation under an anaerobic condition at 25° C. for 7 days.

(3) The glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 0.1 g/L.

(4) The microbial composite reagent obtained in step (3) is added into PFOA-contaminated soil and groundwater at a volume ratio of 1:10 to remove and degrade PFOA, wherein an initial concentration of PFOA in the contaminated soil and groundwater is 1 mg/L.

### Example 2

This example provides a method for removing perfluorooctanoic acid from soil and groundwater, which is different from the method in Example 1 only in that the glucose-based carbonaceous material in the microbial composite reagent is different, and specifically, the temperature of pyrolysis in step (1) is different. In the present example, the temperature of pyrolysis is 700° C.

### Example 3

This example provides a method for removing perfluorooctanoic acid from soil and groundwater, which is different from the method in Example 1 only in that the glucose-based carbonaceous material in the microbial composite reagent is different, and specifically, the temperature of pyrolysis in step (1) is different. In the present example, the temperature of pyrolysis is 1000° C.

### Example 4

This example provides a method for removing perfluorooctanoic acid from soil and groundwater, which is different from the method in Example 1 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 0.5 g/L.

### Example 5

This example provides a method for removing perfluorooctanoic acid from soil and groundwater, which is different from the method in Example 1 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 1 g/L.

### Example 6

This example provides a method for removing perfluorooctanoic acid from soil and groundwater, which is different from the method in Example 2 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 0.5 g/L.

### Example 7

This example provides a method for removing perfluorooctanoic acid from soil and groundwater, which is different from the method in Example 2 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 1 g/L.

### Example 8

This example provides a method for removing perfluorooctanoic acid from soil and groundwater, which is different from the method in Example 3 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 0.5 g/L.

### Example 9

This example provides a method for removing perfluorooctanoic acid from soil and groundwater, which is different from the method in Example 3 only in that the concentration of the glucose-based carbonaceous material in step (3) is different. In the present example, in step (3), the glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 1 g/L.

### Example 10

This example provides a method for removing perfluorooctanoic acid from soil and groundwater, which is different from the method in Example 1 only in that the microbial composite reagent is different, and specifically, the vitamin B in step (2) is different. In the present example, the vitamin B is vitamin B6 instead of vitamin B12.

### Example 11

This example provides a method for removing perfluorooctanoic acid from soil and groundwater, including the following steps:

(1) Under a nitrogen flow rate of 0.5 mL/min, glucose powder is heated to 400° C. at 5° C./min for pyrolysis, and kept at 400° C. for 2 hours to obtain a glucose-based carbonaceous material.

(2) PFOA is added to soil collected from the land of woods and waters at Tsinghua University until the PFOA concentration in the soil reaches 0.05 mg/L, thereby obtaining a PFOA-contaminated soil. 10 g of the PFOA-contaminated soil with the PFOA concentration of 0.05 mg/L is added with 9 g of nutrient broth 022010, 0.5 g of molasses, 0.1 g of vitamin B12, and 500 mL of deionized water, and subjected to microbial acclimation under an anaerobic condition at 20° C. for 5 days.

(3) The glucose-based carbonaceous material is added into the acclimated microorganism solution and subjected to microbial loading under an anaerobic condition for 3 days to obtain a microbial composite reagent of this example. The concentration of the glucose-based carbonaceous material in the microbial composite reagent is 1 g/L.

(4) The microbial composite reagent obtained in step (3) is added into PFOA-contaminated soil and groundwater at a volume ratio of 1:20 to remove and degrade PFOA, wherein an initial concentration of PFOA in the contaminated soil and groundwater is 1 mg/L.

### Example 12

This example provides a method for removing perfluorooctanoic acid from soil and groundwater, including the following steps:

(1) Under a nitrogen flow rate of 2 mL/min, glucose powder is heated to 1000° C. at 15° C./min for pyrolysis, and kept at 1000° C. for 3 hours to obtain a glucose-based carbonaceous material.

(2) PFOA is added to soil collected from the land of woods and waters at Tsinghua University until the PFOA concentration in the soil reaches 0.5 mg/L, thereby obtaining a PFOA-contaminated soil. 10 g of PFOA-contaminated soil with the PFOA concentration of 0.5 mg/L is added with 18 g of nutrient broth 022010, 5 g of molasses, 1 g of vitamin B12, and 500 mL of deionized water and subjected to microbial acclimation under an anaerobic condition at 35° C. for 10 days.

(3) The glucose-based carbonaceous material is added into the acclimated microorganism solution and subjected to microbial loading under an anaerobic condition for 7 days to obtain a microbial composite reagent of this example. The concentration of the glucose-based carbonaceous material in the microbial composite reagent is 5 g/L.

(4) The microbial composite reagent obtained in step (3) is added into PFOA-contaminated soil and groundwater at a volume ratio of 1:10 to remove and degrade PFOA, wherein an initial concentration of PFOA in the contaminated soil and groundwater is 5 mg/L.

### Comparative Example 1

Comparative Example 1 provides a method for removing perfluorooctanoic acid from soil and groundwater, which is different from the method in Example 1 only in that: no glucose-based carbonaceous material is added. The method in Comparative Example 1 is as follows.

(1) PFOA is added to soil collected from the land of woods and waters at Tsinghua University until the PFOA concentration in the soil reaches 0.05 mg/L, thereby obtaining a PFOA-contaminated soil. 10 g of PFOA-contaminated soil with the PFOA concentration of 0.05 mg/L is added with 9 g of nutrient broth 022010, 1 g of molasses, 0.3 g of vitamin B12, and 500 mL of deionized water, and subjected to microbial acclimation under an anaerobic condition at 25° C. for 7 days.

(2) The acclimated microorganism solution obtained in the step (1) is added into PFOA-contaminated soil and groundwater at a volume ratio of 1:10 to remove and degrade PFOA, wherein an initial concentration of PFOA in the contaminated soil and groundwater is 1 mg/L.

### Comparative Example 2

Comparative Example 2 provides a method for removing perfluorooctanoic acid from soil and groundwater, which is different from the method in Example 1 only in that: only the glucose-based carbonaceous material is used in the treatment. The method in Comparative Example 2 is as follows.

(1) Under a nitrogen flow rate of 0.5 mL/min, glucose powder is heated to 400° C. at 5° C./min for pyrolysis, and kept at 400° C. for 3 hours to obtain a glucose-based carbonaceous material.

(2) The glucose-based carbonaceous material obtained in step (1) is added into PFOA-contaminated soil and groundwater at a concentration of 0.1 g/L to remove and degrade PFOA, wherein an initial concentration of PFOA in the contaminated soil and groundwater is 1 mg/L.

### Comparative Example 3

Comparative Example 3 provides a method for removing perfluorooctanoic acid from soil and groundwater, which is different from the method in Example 1 only in that the glucose-based carbonaceous material in the microbial composite reagent is different, and specifically, the temperature of pyrolysis in step (1) is different. In Comparative Example 3, the temperature of pyrolysis is 300° C.

### Comparative Example 4

Comparative Example 4 provides a method for removing perfluorooctanoic acid from soil and groundwater, which is different from the method in Example 1 only in that the glucose-based carbonaceous material in the microbial composite reagent is different, and specifically, the temperature of pyrolysis in step (1) is different. In Comparative Example 4, the temperature of pyrolysis is 1100° C.

### Comparative Example 5

Comparative Example 5 provides a method for removing perfluorooctanoic acid from soil and groundwater, which is different from the method in Example 1 only in that the microbial composite reagent is different. Specifically, no molasses is added in step (2). That is, in step (2) in Comparative Example 5, PFOA is added to soil collected from the land of woods and waters at Tsinghua University until the PFOA concentration in the soil reaches 0.05 mg/L, thereby obtaining a PFOA-contaminated soil. 10 g of PFOA-contaminated soil with the PFOA concentration of 0.05 mg/L is added with 9 g of nutrient broth022010, 0.3 g of vitamin B12, and 500 mL of deionized water, and subjected to microbial acclimation under an anaerobic condition at 25° C. for 7 days.

### Comparative Example 6

Comparative Example 6 provides a method for removing perfluorooctanoic acid from soil and groundwater, which is different from the method in Example 1 only in that the microbial composite reagent is different. Specifically, no vitamin is added in step (2). That is, in step (2) in Comparative Example 6, PFOA is added to soil collected from the land of woods and waters at Tsinghua University until the PFOA concentration in the soil reaches 0.05 mg/L, thereby obtaining a PFOA-contaminated soil. 10 g of PFOA-contaminated soil with the PFOA concentration of 0.05 mg/L is added with 9 g of nutrient broth022010, 1 g of molasses, and 500 mL of deionized water, and subjected to microbial acclimation under an anaerobic condition at 25° C. for 7 days.

The specific tests are described below.

### 1. Characterization of Glucose-Based Carbonaceous Materials

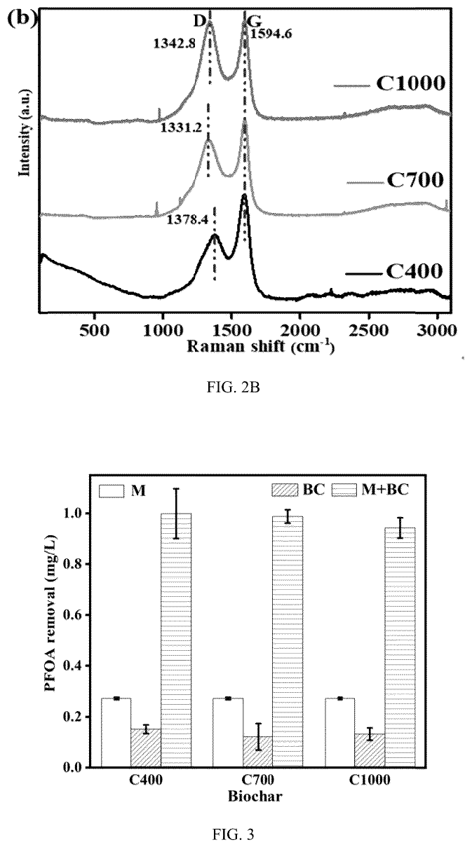

FIGS. 2A and 2B show the XRD and Raman spectra of the glucose-based carbonaceous materials prepared in steps (1) of Examples 1 to 3, wherein FIG. 2A shows the XRD spectra and FIG. 2B shows the Raman spectra. In FIGS. 2A and 2B, “C400”, “C700”, and “C1000” respectively denote the glucose-based carbonaceous materials obtained at the pyrolysis temperatures of 400° C., 700° C., and 1000° C., respectively corresponding to the glucose-based carbonaceous materials prepared in Example 1, Example 2, and Example 3.

The data of specific surface area and intensity ratio of the D peak to the G peak in the Raman spectra of the glucose-based carbonaceous materials prepared in step (1) of Examples 1 to 3 are shown in Table 1 below:

TABLE 1

| | Specific | |
|---|---|---|
| | surface area | |
| | (m²/g) | I_{D}/I_{G} |
| Example 1 | 21.596 | 0.63 |
| Example 2 | 10.817 | 0.77 |
| Example 3 | 105.719 | 1.20 |

### 2. PFOA Removal Effect

The concentrations of PFOA and fluoride ions in the reaction systems are determined by using high performance liquid chromatography-mass spectrometry (HPLC-MS).

FIG. 3 compares PFOA removal rates after treating PFOA-contaminated soil and groundwater for 40 days with: the microorganisms (M) alone in Comparative Example 1, the glucose-based carbonaceous material (BC) alone in Comparative Example 2, and the microbial composite reagents (M+BC) in Examples 1 to 3. In FIG. 3, “C400”, “C700”, and “C1000” respectively denote the glucose-based carbonaceous materials prepared at 400° C., 700° C., and 1000° C., respectively corresponding to the glucose-based carbonaceous materials prepared in Example 1, Example 2, and Example 3. The initial concentration of PFOA in the contaminated soil and groundwater is 1 mg/L.

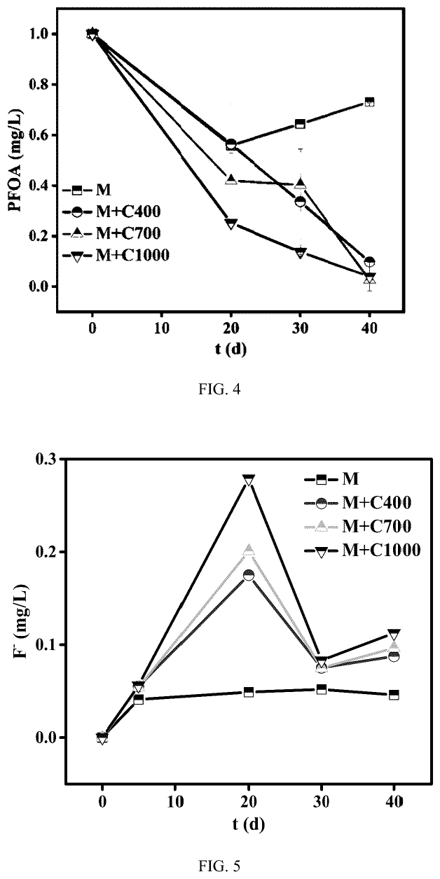

FIG. 4 shows concentration change curves of PFOA in the reaction systems with different treatment times and different glucose-based carbonaceous materials obtained at different pyrolysis temperatures, under the action of the microorganisms alone (M) in Comparative Example 1 and under the actions of the microbial composite reagents (M+C400, M+C700, M+C1000) in Examples 1 to 3. FIG. 5 shows concentration change curves of fluoride ions in the reaction systems with different treatment times and different glucose-based carbonaceous materials obtained at different pyrolysis temperatures, under the action of the microorganisms alone (M) in Comparative Example 1 and under the actions of the microbial composite reagents (M+C400, M+C700, M+C1000) in Examples 1 to 3.

As can be seen from FIGS. 3 and 4, when the microorganisms act alone, the PFOA removal rate is 27.2%, which is relatively low, and the concentration of PFOA decreases continuously in the first 20 days of reaction. However, the concentration of PFOA increases after day 20, which suggests that the microorganisms alone cannot effectively remove PFOA. When the glucose-based carbonaceous materials act alone, the removal rates of the reaction systems with the glucose-based carbonaceous materials prepared at the pyrolysis temperatures of 400° C., 700° C. and 1000° C. are about 15%, which is relatively low. Under the action of the microbial composite reagents of Examples 1 to 3, 94.3% to 99.9% of PFOA is removed after 40 days of reaction, while fluoride ions, as the main degradation product, are detected in relatively high concentrations as shown in FIG. 5, which all suggest that the reaction systems with the glucose-based carbonaceous materials loaded with the microorganisms can degrade the PFOA effectively, and almost all PFOA in the contaminated system can be removed.

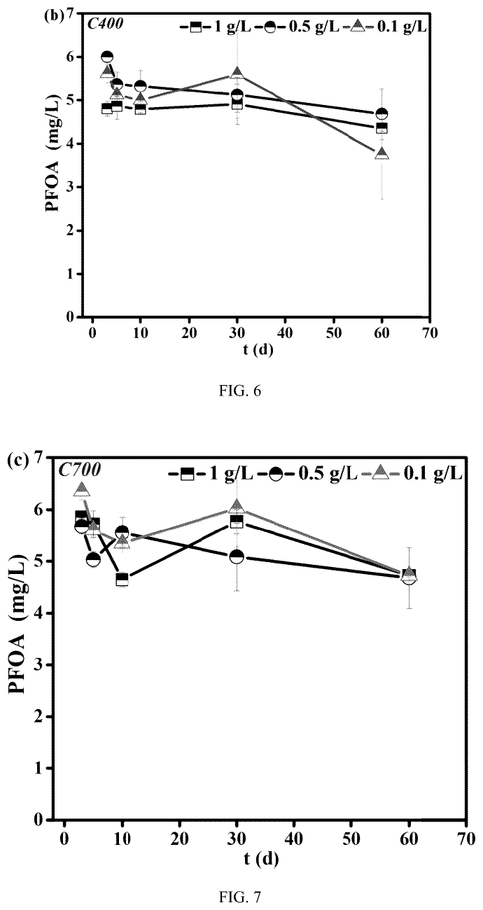

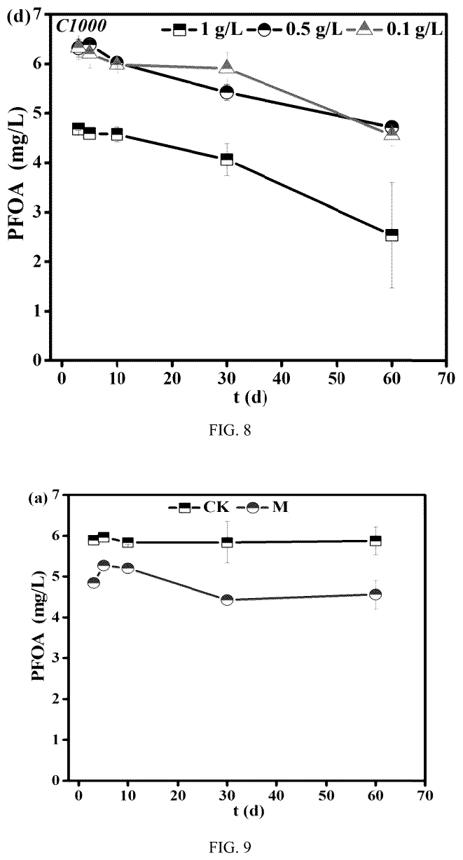

FIG. 6 compares PFOA removal effects of the microbial composite reagents obtained in steps (3) in Example 1, Example 4, and Example 5. In FIG. 6, “0.1 g/L”, “0.5 g/L”, and “1 g/L” respectively denote the concentrations of the glucose-based carbonaceous materials in the microbial composite reagents, respectively corresponding to the microbial composite reagents prepared in Example 1, Example 4, and Example 5. FIG. 7 compares PFOA removal effects of the microbial composite reagents obtained in Example 2, Example 6, and Example 7, step (3). In FIG. 7, “0.1 g/L”, “0.5 g/L”, and “1 g/L” respectively denote the concentrations of the glucose-based carbonaceous materials in the microbial composite reagents, respectively corresponding to the microbial composite reagents prepared in Example 2, Example 6, and Example 7. FIG. 8 compares PFOA removal effects of the microbial composite reagents obtained in steps (3) in Example 3, Example 8, and Example 9. In FIG. 8, “0.1 g/L”, “0.5 g/L”, and “1 g/L” respectively denote the concentrations of the glucose-based carbonaceous materials in the microbial composite reagents, respectively corresponding to the microbial composite reagents prepared in Example 3, Example 8, and Example 9. As can be seen from FIGS. 6 to 8, all microbial composite reagents with the concentrations of the glucose-based carbonaceous material in a range from 0.1 g/L to 1 g/L can achieve good removal effects.

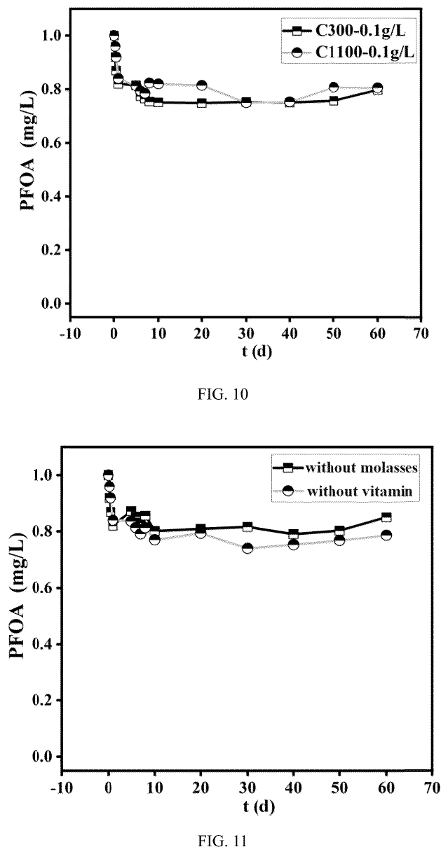

FIG. 9 compares PFOA removal effects of the microorganisms alone in Comparative Example 1 and a blank control with neither microorganisms nor the glucose-based carbonaceous materials. In FIG. 9, “M” denotes Comparative Example 1 and “CK” denotes the blank control without the microorganisms. As can be seen from FIG. 9, the PFOA removal effect under the action of the microorganisms alone is poor and is comparable to that of the blank control.

FIG. 10 compares PFOA removal effects of the microbial composite reagents prepared in steps (3) in Comparative Example 3 and Comparative Example 4. In FIG. 10, “C300-0.1 g/L” denotes that the pyrolysis temperature during the preparation of the glucose-based carbonaceous material is 300° C., while the concentration of the glucose-based carbonaceous material in the microbial composite reagent is 0.1 g/L, corresponding to the microbial composite reagent in Comparative Example 3. “C1100-0.1 g/L” denotes that the pyrolysis temperature during the preparation of the glucose-based carbonaceous material is 1100° C., while the concentration of the glucose-based carbonaceous material in the microbial composite reagent is 0.1 g/L, corresponding to the microbial composite reagent in Comparative Example 4. As can be seen from FIG. 10, when the pyrolysis temperature during the preparation of the glucose-based carbonaceous material is too high or too low, the PFOA removal effect of the microbial composite reagent is relatively poor, and the PFOA concentration after 40 days tends to increase.

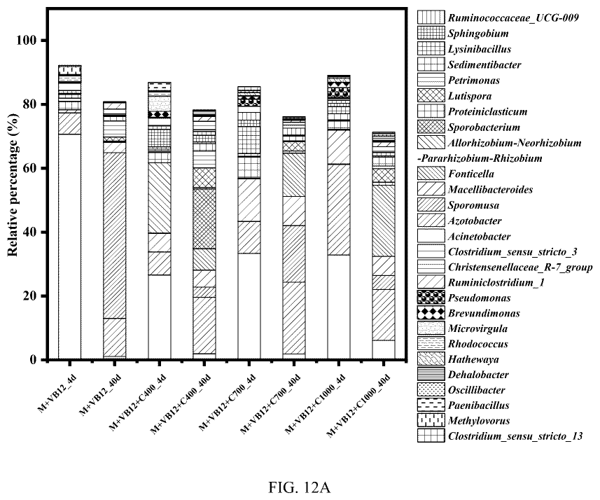

FIG. 11 compares PFOA removal effects of the microbial composite reagents prepared in steps (3) in Comparative Example 5 and Comparative Example 6. It can be seen from this figure that when the microbial composite reagent does not include molasses or the vitamin, the PFOA removal effect is not significant, and the PFOA concentration after 40 days tends to increase.

### 3. Microbial Community Structure of the Reaction System

The microbial diversity and community structure before and after the reaction are investigated using the high-throughput 16s rDNA sequencing technology.

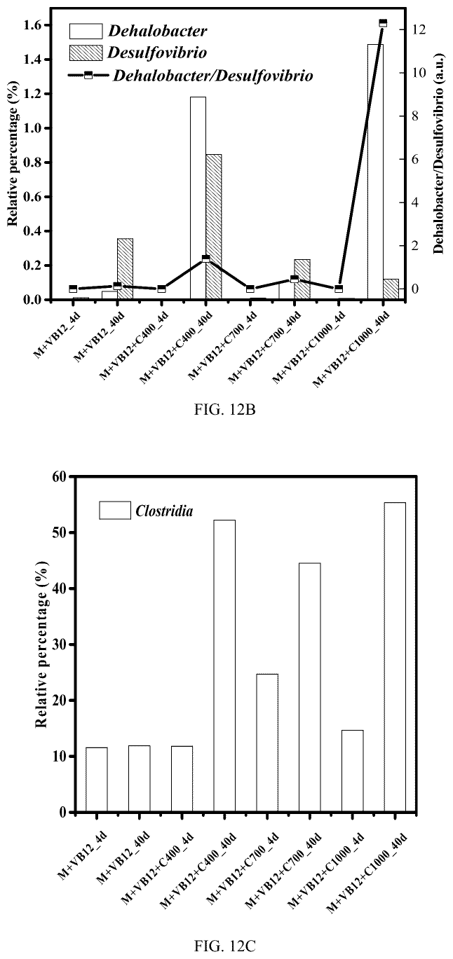

FIGS. 12A to 12 C show the microbial community structure and relative abundances in the systems after 4 days and 40 days under the actions of microorganisms alone (M) in Comparative Example 1 and the microbial composite reagents obtained in steps (3) of Example 1, Example 2, and Example 3. In FIG. 12, “M+VB12” corresponds to Comparative Example 1, and “M+VB12+C400”, “M+VB12+C700”, and “M+VB12+C1000” respectively correspond to Example 1, Example 2, and Example 3.

It can be seen from FIG. 12 that the introduction of the glucose-based carbonaceous material leads to the changes in microbial diversity and community structure. First, as shown in FIG. 12B, the relative abundances of Dehalobacter as specialized dehalogenating bacteria and Desulfovibrio as facultative dehalogenating bacteria are significantly increased after the introduction of the glucose-based carbonaceous material, suggesting that the introduction of the glucose-based carbonaceous material facilitates the growth of the dehalogenating bacteria, thereby promoting the reductive dehalogenation of PFOA. Second, with the progression of the dehalogenation process of PFOA, the proportions of Dehalobacter and Desulfovibrio are increased, suggesting that the glucose-based carbonaceous material contributes to the production of higher-performing functional microorganisms for the reductive dehalogenation of PFOA. Third, the glucose-based carbonaceous material also promotes the enrichment of fermentative electroactive microorganisms. As shown in FIG. 12C, with the progression of the dehalogenation process, the glucose-based carbonaceous material promotes the enrichment of Clostridia as fermentative electroactive microorganisms. The fermentative electroactive microorganisms produce a large amount of electrons during their metabolism, which assist functional dehalogenating bacteria in metabolizing PFOA and promote the reductive dehalogenation of PFOA. Additionally, the glucose-based carbonaceous material also promotes the growth of microorganisms such as Sedimentibacter, Clostridium_sensu_stricto_8, Azobacter, and Fonticella. Sedimentibacter is associated with the reductive dehalogenating of microorganisms, while Clostridium_sensu_stricto_8 and Fonticella can promote the growth of functional dehalogenating bacteria by producing H₂ through the degradation of organics. These results suggest that the glucose-based carbonaceous material not only facilitates the enrichment of fermentative electroactive microorganisms thereby producing a large amount of electrons for the metabolism of functional dehalogenating bacteria, but also promotes the growth of hydrogen-producing bacteria, which further enhance the reductive dehalogenation by the functional microorganisms.

The technical features of the above embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims.

What is claimed is:

1. A method for preparing a microbial composite reagent, comprising steps of:
preparing a glucose-based carbonaceous material by pyrolyzing glucose at a temperature in a range from 400° C. to 1000° C. for at least 0.5 hours under a protective atmosphere;
mixing perfluorooctanoic acid-contaminated soil with a basal medium, molasses, a vitamin B, and water to obtain a mixture, and subjecting the mixture to microbial acclimation under an anaerobic condition to obtain an acclimated microorganism solution;
subjecting the glucose-based carbonaceous material to microbial loading by anaerobically mixing the glucose-based carbonaceous material with the acclimated microorganism solution, thereby obtaining the microbial composite reagent.

2. The method of claim 1, wherein in the step of preparing the glucose-based carbonaceous material, the pyrolysis lasts for 2 hours to 3 hours.

3. The method of claim 1, wherein a concentration of perfluorooctanoic acid in the perfluorooctanoic acid-contaminated soil is greater than 0 and less than or equal to 0.5 mg/L.

4. The method of claim 1, wherein corresponding to every 10 parts by mass of the perfluorooctanoic acid-contaminated soil, an amount of the basal medium is in a range from 9 parts by mass to 18 parts by mass, an amount of molasses is in a range from 0.5 parts by mass to 5 parts by mass, an amount of the vitamin B is in a range from 0.1 parts by mass to 1 part by mass, and an amount of water is in a range from 500 parts by mass to 1000 parts by mass.

5. The method of claim 1, wherein the vitamin B comprises vitamin B12, and/or the basal medium comprises a nutrient broth.

6. The method of claim 1, wherein the microbial acclimation lasts for 5 days to 10 days, and a temperature of the microbial acclimation is in a range from 20° C. to 35° C.

7. The method of claim 1, wherein a concentration of the glucose-based carbonaceous material in the microbial composite reagent is in a range from 0.1 g/L to 1 g/L.

8. The method of claim 1, wherein the perfluorooctanoic acid-contaminated soil comprises microorganisms of Dehalobacter and/or Desulfovibrio.

9. The method of claim 8, wherein the perfluorooctanoic acid-contaminated soil further comprises microorganisms of Sedimentibacter, Clostridium_sensu_stricto_8, Azobacter, Fonticella, or any combinations thereof.

10. A microbial composite reagent, comprising a glucose-based carbonaceous material and an acclimated microorganism solution, wherein the microorganism solution comprises perfluorooctanoic acid-contaminated soil, a basal medium, molasses, a vitamin B, and water;
wherein a specific surface area of the glucose-based carbonaceous material is in a range from 10.817 m²/g to 105.719 m²/g, and an intensity ratio of the D peak to the G peak in a Raman spectrum of the glucose-based carbonaceous material is in a range from 0.63 to 1.20.

11. The microbial composite reagent of claim 10, wherein a concentration of the glucose-based carbonaceous material in the microbial composite reagent is in a range from 0.1 g/L to 1 g/L.

12. The microbial composite reagent of claim 10, wherein a concentration of perfluorooctanoic acid in the perfluorooctanoic acid-contaminated soil is greater than 0 and less than or equal to 0.5 mg/L.

13. The microbial composite reagent of claim 10, wherein corresponding to every 10 parts by mass of the perfluorooctanoic acid-contaminated soil, an amount of the basal medium is in a range from 9 parts by mass to 18 parts by mass, an amount of the molasses is in a range from 0.5 parts by mass to 5 parts by mass, an amount of the vitamin B is in a range from 0.1 parts by mass to 1 part by mass, and an amount of the water is in a range from 500 parts by mass to 1000 parts by mass.

14. The microbial composite reagent of claim 10, wherein the vitamin B comprises vitamin B12, and/or the basal medium comprises a nutrient broth.

15. The microbial composite reagent of claim 10, wherein the perfluorooctanoic acid-contaminated soil comprises microorganisms of Dehalobacter and/or Desulfovibrio.

16. The microbial composite reagent of claim 15, wherein the perfluorooctanoic acid-contaminated soil further comprises microorganisms of Sedimentibacter, Clostridium_sensu_stricto_8, Azobacter, Fonticella, or any combinations thereof.

17. A method for removing perfluorooctanoic acid, comprising a step of:
treating a pollution source with a microbial composite reagent to remove perfluorooctanoic acid from the pollution source;
wherein the microbial composite reagent is prepared by the method of claim 1.

18. The method of claim 17, wherein an initial concentration of the perfluorooctanoic acid in the pollution source is in a range from 0.1 mg/L to 5 mg/L.

19. The method of claim 17, wherein the pollution source comprises contaminated soil and/or contaminated groundwater, and a volume ratio of the microbial composite reagent to the pollution source is in a range from 1:10 to 1:20.

20. The method of claim 17, wherein a time period of treating the pollution source with the microbial composite reagent is greater than 20 days.
